(19) United States
(12) Patent Application Publication
(10) Pub. No.: US 2013/0137912 A1
(43) Pub. Date: May 30, 2013

(54) CATALYTIC COMPOSITION CONTAINING AN ACID FUNCTION AND A PROCESS FOR THE SELECTIVE DIMERIZATION OF ISOBUTENE

(71) Applicant: IFP Energies nouvelles, Rueil-Malmaison Cedex (FR)

(72) Inventors: Lionel MAGNA, Lyon (FR); Helene OLIVIER-BOURBIGOU, Saint Genis-Laval (FR)

(73) Assignee: IFP ENERGIES NOUVELLES, RUEIL-MALMAISON CEDEX (FR)

(21) Appl. No.: 13/688,308

(22) Filed: Nov. 29, 2012

(30) Foreign Application Priority Data
Nov. 30, 2011 (FR) 11/03.653

(51) Int. Cl. B01J 31/02; C07C 2/08

(52) U.S. Cl. CPC B01J 31/0298; C07C 2/08
USPC 585/514; 585/515; 585/510; 585/329; 502/168; 502/164

(57) ABSTRACT

Catalytic composition comprising at least one non-aqueous ionic liquid medium of general formula Q₁⁺A₁⁻, in which Q₁⁺ represents an organic cation and A₁⁻ represents an anion, and at least one ionic component of general formula Q₂⁺A₂⁻, in which Q₂⁺ represents an organic cation comprising at least one sulphonic acid or carboxylic acid function, and A₂⁻ represents an anion. The invention also relates to an isobutene dimerization process using the catalytic composition.

The present invention relates to the dimerization of isobutene. More particularly a subject of the present invention is a novel catalytic composition and a process for the dimerization of isobutene (pure or in a mixture with other hydrocarbons) using this novel catalytic composition.

It is known that the dimers of isobutene (2,4,4-trimethyl-1-(and -2-) pentene) are useful intermediates for the production of different products of commercial interest. By way of example, the higher alcohols, aldehydes and the acids can be mentioned.

2,2,4-Trimethylpentane can be obtained by hydrogenation of trimethylpentenes and constitutes a sought additive for the reformulation of gasolines (absence of sulphur, aromatics and olefins, and low volatility added to a high octane number: Motor octane number (MON) (sic)=Research octane number (RON)=100).

Thus, the selective dimerization of isobutene, followed by hydrogenation of the obtained products to 2,2,4-trimethylpentane having a high octane number, constitutes a useful route which allows
i) the replacement of MTBE (Methyl-Tert-Butyl-Ether: RON=118; MON=100), which is banned for environmental reasons, and
ii) the use of isobutene, originating from the C4 cuts from catalytic cracking or steam cracking processes, a raw material in the production of MTBE.

The dimerization (and also the oligomerization) of isobutene is an exothermic reaction catalyzed by acids. Different acids have been described in the literature such as sulphuric acid, or derivatives thereof, the chlorinated or fluorinated aluminas, zeolites, silica-aluminas etc. However, those most typically used in industry are phosphoric acid, generally supported, and ion-exchange resins (SP-Isoether process licensed by Snamprogetti or UOP's InAlk process).

The main difficulty associated with these processes is obtaining good dimer selectivity. In fact, the exothermicity of the reaction is often difficult to control and leads to the formation of oligomers (essentially C12 olefins and C16 olefins) obtained by parallel reactions starting from isobutene. These oligomers have boiling points which are too high, and are outside, or at the limit, of the specifications required for reformulated gasolines. Moreover, these oligomers contribute to deactivation of the catalysts.

Different works in the literature describe certain solutions for minimizing the formation of these oligomers.

In the case of the ion-exchange resins (Amberlyst-15 or -35 type), the use of a diluent (or solvent) is often recommended. The dimer selectivity depends on the choice of this solvent. The most efficient additives are the alcohols (U.S. Pat. No. 5,877,372; U.S. Pat. No. 4,100,220), which lead to the co-production of ethers, or ethers (in U.S. Pat. No. 4,447,668, MTBE, ETBE, etc.). Mention may be made of the works by Snamprogetti (M. Marchionna et al. Catal. Today, 65 (2001) 397-403, GB 2 325 237) studying the influence of the addition of MTBE or MeOH with the objective of reusing the existing units of MTBE. Useful trimethylpentene selectivities can thus be achieved but often with less than 85% isobutene conversion.

International patent application WO-A-01/51 435 describes a series of processes in which isobutene is produced by dehydration of tert-butyl alcohol. The isobutene is preferentially dimerized by an Amberlyst A-15® type resin in the presence of tert-butyl alcohol (selectivity promoter) and alkane (butane or isobutane) as diluent. The presence of a hindered alcohol discourages the formation of ether but also reduces the rate of reaction.

All the processes described previously have limitations such as the risks of premature deactivation of the catalyst by “clogging” with the heavier oligomers or also the need to use organic additives to control the selectivity of the reaction, organic additives which are often converted and consumed during the reaction and are therefore not recyclable.

The non-aqueous ionic liquids of composition Q⁺A⁻ have been the subject of several articles (for example, H. Olivier-Bourbigou et al., Appl. Catal. A: General, 2010, 1-56). They find numerous applications as solvents for catalysis by transition metals or as extraction solvents for carrying out liquid-liquid extractions.

The patent application WO-A-00/16902 describes the use of an ionic liquid free of Lewis acidity obtained by reaction of a nitrogen-containing (for example an amine or a quaternary ammonium) or phosphorus-containing compound with a Brønsted acid in quantities such that the ratio of said nitrogen-containing or phosphorus-containing compound to the acid is less than 1. These media are used to catalyze in particular the alkylation of benzene with 1-decene. The Brønsted acids used are those capable of generating anions such as BF₄⁻, PF₆⁻, RCOO⁻ not containing a sulphonic acid or carboxylic acid function.

It has also been described in the application FR2829039 that the addition of at least one Brønsted acid, designated HB, in a non-aqueous liquid medium with an ionic character (“molten salt” type medium) comprising at least one organic cation Q⁺ and an anion A⁻ and in which, when A and B are identical, the molar ratio of the Brønsted acid to the ionic liquid is less than 1/1, leads to liquid compositions which can be used as catalysts and solvents for acid catalysis reactions. In this patent application, it was mentioned that the catalytic composition described could be used more particularly in the dimerization of isobutene.

The use of the ionic liquids as solvents and acid catalysts for the selective dimerization of isobutene has also been described in the U.S. Pat. No. 7,256,152.

The anions B of the acid HB described in FR2829039 and U.S. Pat. No. 7,256,152 are chosen from the following anions: tetrafluoroborates, tetraalkylborates, hexafluorophosphates, hexafluoroantimonates, alkylsulphonates and arylsulphonates, perfluoroalkylsulphonates, fluorosulphonate, sulphates, phosphates, perfluoroacetates, perfluoroalkylsulphonamides, fluorosulphonamides, perfluoroalkylsulphomethides and carboranes.

The advantage of these liquid catalytic systems for the dimerization reaction of isobutene to isooctenes is that they are not very miscible with the reaction products which can therefore be separated by decantation. The catalytic phase can then be recycled and reused, the consumption of catalyst is thus reduced. However, these systems also have limitations. Like heterogeneous catalysts, it is sometimes difficult to maintain a recyclability of the catalytic system while maintaining good selectivity of dimerization products at a high isobutene conversion.

A subject of the present invention is to provide a novel catalytic composition making it possible to obtain a very good recyclability of the catalytic system without a significant lowering of its catalytic activity and without a significant lowering of its dimer selectivity.

More particularly, the present invention relates to a catalytic composition comprising at least one non-aqueous ionic liquid medium of general formula Q₁⁺A₁⁻, in which Q₁⁺ represents an organic cation and A₁⁻ represents an anion, and at least one ionic component of general formula Q₂⁺A₂⁻, in which Q₂⁺ represents an organic cation comprising at least one sulphonic acid or carboxylic acid function, and A₂⁻ represents an anion.

The ionic compounds are defined as being organic acid compounds capable of donating at least one proton.

The ionic liquid Q₁⁺A₁⁻ is defined such that Q₁⁺ represents a quaternary ammonium and/or a quaternary phosphonium and/or a trialkylsulphonium and A₁⁻ represents any anion known to be non-coordinating and capable of forming a liquid salt at low temperature, i.e. below 150° C. The ionic component of general formula Q₂⁺A₂⁻ is defined such that Q₂⁺ represents a quaternary ammonium and/or a quaternary phosphonium and/or a trialkylsulphonium comprising at least one sulphonic acid or carboxylic acid function and A₂⁻ represents any anion known to be non-coordinating and capable of forming a liquid salt at low temperature, i.e. below 150° C.

The anions A₁⁻ or A₂⁻ considered in the invention are preferably chosen from the following anions: tetrafluoroborate, tetraalkylborates, hexafluorophosphate, hexafluoroantimonate, alkylsulphonates and arylsulphonates (for example methylsulphonate or tosylate), perfluoroalkylsulphonates (for example trifluoromethylsulphonate), fluorosulphonate, sulphates, phosphates, perfluoroacetates (for example trifluoroacetate), perfluoroalkylsulphonamides (for example bis-trifluoromethanesulphonyl amide (N(CF₃SO₂)₂), fluorosulphonamides, perfluoroalkylsulphomethides (for example tris-trifluoromethanesulphonyl methylide (C(CF₃SO₂)₃⁻) and carboranes, A₁⁻ and A₂⁻ being identical or different. Preferably, A₁⁻ and A₂⁻ are identical.

The cations Q₁⁺ and Q₂⁺ considered in the invention are preferably chosen from the quaternary ammoniums and/or the quaternary phosphoniums and/or the trialkylsulphoniums. The chemical nature of Q₁⁺ and Q₂⁺ can be identical or different, it is preferably identical. By “identical chemical nature” is meant that if, for example, the cation Q₁⁺ is a quaternary ammonium, Q₂⁺ is also a quaternary ammonium. By “different chemical nature” is meant that if, for example, the cation Q₁⁺ is a quaternary ammonium, Q₂⁺ is chosen from a quaternary phosphonium or a trialkylsulphonium. In any case, the organic cation Q₂⁺ of the composition Q₂⁺A₂⁻ contains at least one sulphonic acid or carboxylic acid function, while the substituents of the organic cation Q₁⁺ do not have such a function. By “sulphonic acid or carboxylic acid function” is meant a hydrocarbyl substituent having 1 to 12 carbon atoms containing a sulphonic acid (—SO₃H) or carboxylic acid (—CO₂H) group grafted onto the cation Q₂⁺.

The quaternary ammoniums and/or phosphoniums of general formula Q₁⁺ and Q₂⁺ preferably correspond to the formulae NR¹R²R³R⁴⁺ and PR¹R²R³R⁴⁺, or to general formulae R¹R²N═CR³R⁴⁺ and R¹R²P═CR³R⁴⁺ where
for Q₁⁺: R¹, R², R³ and R⁴, identical or different, represent hydrogen, with the exception of the cation NH₄⁺ for NR¹R²R³R⁴⁺, and preferably a single substituent can represent the hydrogen atom, or hydrocarbyl radicals having 1 to 12 carbon atoms, for example alkyl groups, saturated or unsaturated, cycloalkyls or aromatics, aryl or aralkyl, comprising from 1 to 12 carbon atoms,
and for Q₂⁺: at least one substituent R¹, R², R³ or R⁴ represents a hydrocarbyl radical having 1 to 12 carbon atoms containing at least one sulphonic acid or carboxylic acid the substituents not having a sulphonic acid (—SO₃H) or carboxylic acid (—CO₂H) function, identical or different, are defined as previously for Q₁⁺,

The quaternary ammoniums and/or phosphoniums of general formula Q₁⁺ and Q₂⁺ can also be derived from nitrogen-containing (imidazolium, pyridinium, pyrrolidinium, pyrazolium, triazolium) or phosphorus-containing heterocycles comprising 1, 2 or 3 nitrogen and/or phosphorus atoms, of general formulae:

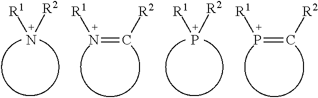

in which the rings are constituted by 4 to 10 atoms, preferably 5 to 6 atoms, R¹ and R² for Q₁⁺ or Q₂⁺ being defined as previously.

The quaternary ammoniums and/or phosphoniums of general formula Q₁⁺ and Q₂⁺ can also consist of a cation corresponding to one of general formulae:

R¹R²±N═CR³—R⁵—R³C═N⁺R¹R² and

R¹R²⁺P═CR³—R⁵—R³C═P⁺R¹R²

in which R¹, R² and R³, identical or different, are defined as previously for Q₁⁺ or Q₂⁺ and R⁵ represents an alkylene or phenylene radical.

Among the R¹, R², R³ and R⁴ groups the methyl, ethyl, propyl, isopropyl, butyl, sec-butyl, tert-butyl, amyl, phenyl or benzyl radicals will be mentioned; R⁵ can be a methylene, ethylene, propylene or phenylene group.

Among the R¹, R², R³ and R⁴ groups containing a sulphonic acid or carboxylic acid function the —CH₂SO₃H, —(CH₂)₂SO₃H, —(CH₂)₃SO₃H, —(CH₂)₄SO₃H, —C(CH₃)₂—CH₂SO₃H, —CH₂—C(CH₃)₂SO₃H, —C(CH₃)H—CH₂SO₃H, —C₆H₄—CH₂SO₃H, —CH₂CO₂H, —(CH₂)₂CO₂H, —(CH₂)₃CO₂H, —(CH₂)₄CO₂H, —C(CH₃)₂—CH₂CO₂H, —CH₂—C(CH₃)₂CO₂H, —C(CH₃)H—CH₂CO₂H, —C₆H₄—CH₂CO₂H radicals will be mentioned.

The ammonium and/or phosphonium cation Q₁⁺ is preferably chosen from the group formed by N-butylpyridinium, N-ethylpyridinium, 1-butyl-3-methylimidazolium, diethylpyrazolium, 1-ethyl-3-methylimidazolium, pyridinium, trimethylphenylammonium, tetrabutylphosphonium, N-ethyl-N-methylpyrrolidinium and N-butyl-N-ethylpyrrolidinium.

The ammonium and/or phosphonium cation Q₂⁺ is preferably chosen from the group formed by 1-butyl-3-(2-ethylsulphonic)imidazolium, 1-ethyl-3-(2-ethylcarboxylic)imidazolium, N-butyl-N-(2-ethylsulphonic)pyrrolidinium, N-ethyl-N-(2-ethylcarboxylic)pyrrolidinium, (2-ethylsulphonic)triethylammonium and triphenyl(3-propylsulphonic)phosphonium.

The trialkylsulphoniums of general formula Q₁⁺ or Q₂⁺ considered in the invention have the general formula SR¹R²R³⁺ in which
for Q₁⁺: R¹, R² and R³ identical or different, represent hydrocarbyl radicals having 1 to 12 carbon atoms, for example alkyl or alkenyl, cycloalkyl or aromatic, aryl or aralkyl groups, comprising from 1 to 12 carbon atoms,
and for Q₂⁺: at least one substituent R¹, R² or R³ represents a hydrocarbyl radical having 1 to 12 carbon atoms containing at least one sulphonic acid or carboxylic acid function, the substituents not having a sulphonic acid or carboxylic acid function, identical or different, are defined as previously for Q₁⁺.

As examples of the ionic liquids Q₁⁺A₁⁻ which can be used, N-butylpyridinium hexafluorophosphate, N-ethyl pyridinium tetrafluoroborate, 1-butyl-3-methylimidazolium hexafluoroantimonate, 1-butyl-3-methylimidazolium hexafluorophosphate, 1-butyl-3-methylimidazolium trifluoromethylsulphonate, pyridinium fluorosulphonate, trimethylphenylammonium hexafluorophosphate, 1-butyl-3-methylimidazolium bis-trifluoromethylsulphonylamide, N-ethyl-N-methylpyrrolidinium bis-trifluoromethylsulphonylamide, triethylsulphonium bis-trifluoromethylsulphonylamide, tributylhexylammonium bis-trifluoromethylsulphonylamide, 1-butyl-3-methylimidazolium trifluoroacetate and 1-butyl-2,3-dimethylimidazolium bis-trifluoromethylsulphonylamide can be mentioned.

These ionic liquids can be used alone or in a mixture. They have a solvent function.

As examples of the compositions Q₂⁺A₂⁻ which can be used, 1-methyl-3-(2-ethylsulfonic)imidazolium trifluoromethylsulphonate, 1-ethyl-3-(2-ethylcarboxylic)imidazolium bistriflylamide, N-butyl-N-(2-ethylsulphonic)pyrrolidinium trifluoromethylsulphonate, N-ethyl-N-(2-ethylcarboxylic)pyrrolidinium bistriflylamide, (2-ethylsulphonic)triethylammonium trifluoromethylsulphonate and triphenyl(3-propylsulphonic)phosphonium paratoluene sulphonate can be mentioned.

These compositions can be used alone or in mixture.

The use of an ionic component of formula Q₂⁺A₂⁻ as defined in the catalytic composition of the present invention makes it possible to promote the immobilization of the acid in the ionic liquid by grafting of the (sulphonic or carboxylic) acid function onto the organic cation Q₂⁺ component of the acid. Therefore, due to a similar chemical structure, the acid is perfectly immobilized in the ionic liquid Q₁⁺A₁⁻ which allows an excellent recyclability of the catalytic composition to be obtained.

Preferably, the anions A₁⁻ and A₂⁻ are identical in the catalytic composition used according to the invention. The molar ratio of the ionic component Q₂⁺A₂⁻ to the ionic liquid Q₁⁺A₁⁻ is preferably less than 5/1, and yet more preferably less than 2/1.

The ionic liquid Q₁⁺A₁⁻ makes it possible to dissolve the ionic component Q₂⁺A₂⁻ which in itself is very viscous. In this way control of the level of the acidity in the catalytic composition is obtained as well as the implementation of a biphasic system in the presence of the reagents and their products, such as isobutene and its dimers. This allows easy separation (by decantation) and consequently a very good recyclability.

The compounds involved in the catalytic composition used in the process of the invention can be mixed in any order. The mixing can be done by simply bringing them into contact followed by stirring until a homogeneous liquid is formed. This mixing can be done outside the reactor used for the catalytic application or inside this reactor.

Also a subject of the present invention is an isobutene dimerization process using the catalytic composition. The dimerization process according to the invention applies to pure isobutene or isobutene in a mixture with other hydrocarbons.

The sources of isobutene are varied. However, the most common are the dehydrogenation of isobutane and the dehydration of tert-butyl alcohol The isobutene can also originate from a C4 cut from catalytic cracking in a fluidized bed or from steam cracking.

In this latter case, the isobutene can be used in a mixture with n-butenes, isobutane and butane. The process according to the present invention then has the additional advantage of making it possible to selectively convert the isobutene without having to separate the other constituents of the cut. Another advantage of the process according to the invention is that the isobutene-butene co-dimerization can be limited.

Recent advances in the field of biotechnologies show that it is also possible to produce isobutene from 2-methyl-propanol (isobutanol), itself obtained from sugars originating from the fermentation of biomass.

The ratio by volume of the isobutene to the catalytic composition can be comprised between 0.1/1 and 1000/1, preferably between 1/1 and 100/1. It is chosen so as to obtain the best selectivities.

The process can be carried out in a closed system, in a semi-open system or continuously with one or more reaction stages. At the reactor outlet, the organic phase containing the reaction products is separated.

In the dimerization process of the invention, an organic solvent such as an aliphatic hydrocarbon or an aromatic hydrocarbon immiscible or partially miscible with the ionic liquid can be added to the catalytic composition which allows a better separation of the phases. As aliphatic hydrocarbon for example pentane, heptane, cyclohexane, decane, dodecane or a paraffinic feedstock can be used, alone or in a mixture. As aromatic hydrocarbon for example toluene or xylene can be used, alone or in a mixture.

The temperature at which the dimerization reaction is carried out ranges for example from −50° C. to 200° C.; it is advantageously below 100° C.

The reaction can be carried out at autogeneous pressure, this can also be increased up to 10 MPa.

The dimerization reaction can be carried out using a reactive distillation technique.

The products obtained by the present invention can be subsequently converted according to different reactions, such as hydrogenation, hydroformylation, oxidation, etherification, epoxidation or hydration.

The following examples illustrate the invention without limiting its scope.

### EXAMPLE 1 (ACCORDING TO THE INVENTION)

### Preparation of the Acid [MeIM(CH₂)₄SO₃H][CF₃SO₃]

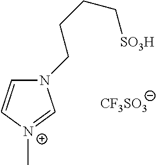

9.12 g (0.067 mmol) of 1,4-butanesultone dissolved in 150 mL of toluene is introduced under an inert atmosphere into a 250 mL flask. 5.469 g (0.067 mmol) of 1-methylimidazole is added slowly. The mixture is stirred at ambient temperature for 1 h then under reflux for 12 h. After returning to ambient temperature, the solid formed is filtered then washed with toluene and diethyl ether. It is finally dried under vacuum at ambient temperature (yield >90%). This compound is suspended in 100 mL of dry toluene. Then 1 molar equivalent of triflic acid is added slowly. After reaction for 1 h at ambient temperature, the toluene is eliminated under dynamic vacuum. The liquid residue is finally dried at 80° C. under dynamic vacuum. [MeIm(CH₂)₄SO₃H][CF₃SO₃] is obtained with a quasi-quantitative yield. ¹H and ¹³C NMR analyses confirm the structure of the expected product.

### EXAMPLE 2 (ACCORDING TO THE INVENTION)

### Preparation of the Ionic Liquid [BMIm][CF₃SO₃] and Acid [MeIm(CH₂)₄SO₃H][CF₃SO₃] Catalytic System

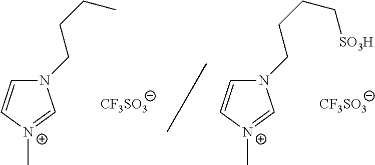

4.37 g (0.012 mol) of [MeIm(CH₂)₄SO₃H][CF₃SO₃] described in Example 1 is mixed at ambient temperature, under an inert atmosphere, with 2 mL of [BMIm][CF₃SO₃]. The mixture represents a volume of 5 mL. The mixture is stirred for a few minutes and leads to a clear solution containing 2.4 mol/L of acid.

### EXAMPLE 3 (ACCORDING TO THE INVENTION)

### Dimerization of Isobutene Using the Catalytic Composition of Example 2

All of the mixture prepared in Example 2 is introduced, under an argon atmosphere, into a 100 mL Fisher-Porter tube, provided with a magnetic stirring bar and dried before the study and held under vacuum, Then, 20 mL (11.2 g) of a liquid feedstock containing 95% isobutene and 5% of n-butane is introduced, at ambient temperature. Stirring is then started (reaction time zero). After reaction for 20 minutes at 25° C., the stirring is stopped. The supernatant organic phase is separated from the ionic liquid phase and analyzed by GC (gas chromatography, with heptane as external standard) after treatment with soda (10N) in order to remove possible traces of acid and drying over MgSO₄. The conversion of isobutene is 71%. The dimerization product selectivity (C8) is 86%, the trimer selectivity (C12) is 14%, the tetramer selectivity (C16) is less than 1%.

The ionic liquid phase containing the “[MeIm(CH₂)₄SO₃H][CF₃SO₃]/[BMIm][CF₃SO₃]” system was isolated and reused over several catalysis cycles without adjustment of the catalytic composition (Examples 4 to 13). The results obtained are shown in the table.

TABLE
Recycling of the “[BMIm][CF₃SO₃]/[MeIm(CH₂)₄SO₃H][CF₃SO₃]” system

| | | | | Conv. | Selectivities (wt. %) | | |
|---|---|---|---|---|---|---|---|
| Ex | Ionic liquid | Nature of added acid | mol | (wt. %) | C8 | C12 | C16 |
| 3 | [BMIm][CF₃SO₃] | [MeIm(CH₂)₄SO₃H] [CF₃SO₃] | 0.012 | 71 | 86 | 14 | <1 |

| | | | | | |
|---|---|---|---|---|---|
| 4 | Recycling 1 | 71 | 87 | 12 | <1 |
| 5 | Recycling 2 | 71 | 88 | 12 | <1 |
| 6 | Recycling 3 | 66 | 88 | 12 | <1 |
| 7 | Recycling 4 | 64 | 88 | 12 | <1 |
| 8 | Recycling 5 | 68 | 88 | 11 | <1 |
| 9 | Recycling 6 | 60 | 89 | 10 | <1 |
| 10 | Recycling 7 | 67 | 89 | 11 | <1 |
| 11 | Recycling 8 | 61 | 89 | 11 | <1 |
| 12 | Recycling 9 | 66 | 90 | 10 | <1 |
| 13 | Recycling 10 | 68 | 90 | 10 | <1 |

Comparison of the proton NMR spectra of the ionic liquid phase before and after this recycling series demonstrates the perfect immobilization of the acid in the ionic liquid. The catalytic composition can thus be recycled without a significant lowering of its catalytic activity and without a significant lowering of its dimer selectivity.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding FR application Ser. No. 11/03.653, filed 30 Nov. 2011, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Catalytic composition comprising at least one non-aqueous ionic liquid medium of general formula Q₁⁺A₁⁻, in which Q₁⁺ represents an organic cation and A₁⁻ represents an anion, and at least one ionic component of general formula Q₂⁺A₂⁻, in which Q₂⁺ represents an organic cation comprising at least one sulphonic acid or carboxylic acid function, and A₂⁻ represents an anion.

2. Catalytic composition according to claim 1, in which the anion A₁⁻ or A₂⁻ is chosen from the following anions: tetrafluoroborate, tetraalkylborates, hexafluorophosphate, hexafluoroantimonate, alkylsulphonates, arylsulphonates, perfluoroalkylsulphonates, fluorosulphonate, sulphates, phosphates, perfluoroacetates, perfluoroalkylsulphonamides, fluorosulphonamides, perfluoroalkylsulphomethides and carboranes, A₁⁻ and A₂⁻ being identical or different.

3. Catalytic composition according to claim 1, in which Q₁⁺ represents a quaternary ammonium and/or a quaternary phosphonium and/or a trialkylsulphonium, and Q₂⁺ represents a quaternary ammonium and/or a quaternary phosphonium and/or a trialkylsulphonium comprising at least one sulphonic acid or carboxylic acid function, and A₁⁻ or A₂⁻ represent any anion known to be non-coordinating and capable of forming a liquid salt below 150° C.

4. Catalytic composition according to claim 3, in which the ammonium and/or quaternary phosphonium cation Q₁⁺ or Q₂⁺ is chosen from:
the ammonium and/or quaternary phosphonium cations corresponding to one of the general formulae NR¹R²R³R⁴⁺ and PR¹R²R³R⁴⁺, or to one of the general formulae R¹R²N═CR³R⁴⁺ and R¹R²P═CR³R⁴⁺ where,
for Q₁⁺: R¹, R², R³ and R⁴, identical or different, represent hydrogen, with the exception of the cation NH₄⁺ for NR¹R²R³R⁴⁺, a single substituent representing the hydrogen atom, or hydrocarbyl radicals having 1 to 12 carbon atoms,
and for Q₂⁺: at least one substituent R¹, R² or R³ represents a hydrocarbyl radical having 1 to 12 carbon atoms containing at least one sulphonic acid or carboxylic acid function, the substituents not having a sulphonic acid or carboxylic acid function, identical or different, are defined as previously for Q₁⁺,
the quaternary ammonium and/or phosphonium cations derived from nitrogen-containing or phosphorus-containing heterocycles comprising 1, 2 or 3 nitrogen and/or phosphorus atoms, of general formulae: 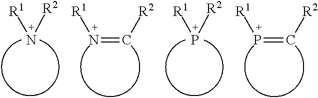
in which the rings are constituted by 4 to 10 atoms, preferably 5 to 6 atoms, R¹ and R² for Q₁⁺ or Q₂⁺ being defined as previously,
the quaternary ammonium and/or phosphonium cations corresponding to one of general formulae:
R¹R²⁺N═CR³—R⁵—R³C═N⁺R¹R²
R¹R²⁺P═CR³—R⁵—R³C═P⁺R¹R²
in which R¹, R² and R³, identical or different, are defined as previously for Q₁⁺ or Q₂⁺ and R⁵ represents an alkylene or phenylene radical.

5. Catalytic composition according to claim 4, in which the quaternary ammonium and/or phosphonium cation Q₁⁺ is chosen from the group formed by N-butylpyridinium, N-ethylpyridinium, 1-butyl-3-methylimidazolium, diethylpyrazolium, 1-ethyl-3-methylimidazolium, pyridinium, trimethylphenylammonium, tetrabutylphosphonium, N-ethyl-N-methylpyrrolidinium and N-butyl-N-ethylpyrrolidinium.

6. Catalytic composition according to claim 4, in which the ammonium and/or phosphonium cation Q₂⁺ is chosen from the group formed by 1-butyl-3-(2-ethylsulphonic)imidazolium, 1-ethyl-3-(2-ethylcarboxylic)-imidazolium, N-butyl-N-(2-ethylsulphonic)pyrrolidinium, N-ethyl-N-(2-ethylcarboxylic)pyrrolidinium, (2-ethylsulphonic)triethylammonium and triphenyl(3-propylsulphonic)phosphonium.

7. Catalytic composition according to claim 3, in which Q₁⁺ or Q₂⁺ is a trialkylsulphonium cation corresponding to general formula SR¹R²R³⁺ in which
for Q₁⁺: R¹, R² and R³, identical or different, represent hydrocarbyl radicals having 1 to 12 carbon atoms,
and for Q₂⁺: at least one substituent R¹, R² or R³ represents a hydrocarbyl radical having 1 to 12 carbon atoms containing at least one sulphonic acid or carboxylic acid function, the substituents not having a sulphonic acid or carboxylic acid function, identical or different, are defined as previously for Q₁⁺.

8. Catalytic composition according to claim 1, in which the ionic liquid Q₁⁺A₁⁻ is chosen from N-butylpyridinium hexafluorophosphate, N-ethyl pyridinium tetrafluoroborate, 1-butyl-3-methylimidazolium hexafluoroantimonate, 1-butyl-3-methylimidazolium hexafluorophosphate, 1-butyl-3-methylimidazolium trifluoromethylsulphonate, pyridinium fluorosulphonate, trimethylphenylammonium hexafluorophosphate, 1-butyl-3-methylimidazolium bis-trifluoromethylsulphonylamide, N-ethyl-N-methylpyrrolidinium bis-trifluoromethylsulphonylamide, triethylsulphonium bis-trifluoromethylsulphonylamide, tributylhexylammonium bis-trifluoromethylsulphonylamide, 1-butyl-3-methylimidazolium trifluoroacetate and 1-butyl-2,3-dimethylimidazolium bis-trifluoromethylsulphonylamide, alone or in a mixture.

9. Catalytic composition according to claim 1, in which the ionic component Q₂⁺A₂⁻ is chosen from 1-methyl-3-(2-ethylsulphonic)imidazolium trifluoromethylsulphonate, 1-ethyl-3-(2-ethylcarboxylic)imidazolium bistriflylamide, N-butyl-N-(2-ethylsulphonic)pyrrolidinium trifluoromethylsulphonate, N-ethyl-N-(2-ethylcarboxylic)pyrrolidinium bistriflylamide, (2-ethylsulphonic)triethylammonium trifluoromethylsulphonate and triphenyl(3-propylsulphonic)phosphonium paratoluene sulphonate, alone or in mixture.

10. Catalytic composition according to claim 1, in which the molar ratio of the ionic component Q₂⁺A₂⁻ to the ionic liquid Q₁⁺A₁⁻ is less than 5/1.

11. Isobutene dimerization process using a catalytic composition according to claim 1.

12. Isobutene dimerization process according to claim 11 in which the ratio by volume of the isobutene to the catalytic composition is comprised between 0.1/1 and 1000/1.

13. Isobutene dimerization process according to claim 11 in which an aliphatic hydrocarbon or an aromatic hydrocarbon immiscible or partially miscible with the ionic liquid is added to the catalytic composition.

14. Isobutene dimerization process according to claim 11 in which the dimerization reaction is carried out at a temperature between −50° C. and 200° C., at a pressure ranging from autogeneous pressure to 10 MPa.

15. Isobutene dimerization process according to claim 11 characterized in that it is carried out in a closed system, in a semi-open system or continuously with one or more reaction stages.
